(19) United States
(12) Patent Application Publication
(10) Pub. No.: US 2022/0361906 A1
(43) Pub. Date: Nov. 17, 2022

(54) SURGICAL SYSTEM AND METHOD OF USE

(71) Applicant: Csaba TRUCKAI, Saratoga, CA (US)

(72) Inventor: Csaba TRUCKAI, Saratoga, CA (US)

(21) Appl. No.: 17/662,298

(22) Filed: May 6, 2022

Related U.S. Application Data
(60) Provisional application No. 63/201,725, filed on May 11, 2021.

(51) Int. Cl. A61B 17/32; A61B 34/20; A61B 34/30

(52) U.S. Cl. CPC A61B 17/32; A61B 34/20; A61B 34/30; A61B 2034/2063; A61B 2034/302; A61B 2017/320064; A61B 2017/00199

(57) ABSTRACT

Systems and devices for resecting and removing tissue or organs from the interior of a patient's body, in a minimally invasive laparoscopic procedure while preventing any dispersion of potentially malignant tissue during the resection process.

### CROSS-REFERENCE TO RELATED APPLICATIONS

This application claims the benefit of U.S. Provisional Patent Application No. 63/201,725, filed on May 11, 2021, the content of which is incorporated herein by reference in its entirety.

### FIELD OF THE INVENTION

The present invention relates to systems and devices for resecting and removing tissue or organs from the interior of a patient's body, in a minimally invasive laparoscopic procedure while preventing any dispersion of potentially malignant tissue during the resection process.

### BACKGROUND

The present invention relates to systems and devices for resecting and removing tissue or organs from the interior of a patient's body, in a minimally invasive laparoscopic procedure while preventing any dispersion of potentially malignant tissue during the resection process.

Several surgical procedures require removing a tissue mass or an organ from the body of a patient in an efficient manner preventing dispersion of potentially malignant tissue during the resection process. One such procedures is a hysterectomy where a woman's uterus is detached and removed from her body. Hysterectomy is typically performed in cases of severe endometriosis, presence of fibroids, cancer, cervical dysplasia, uterine prolapse and more. With the advent of minimally invasive surgery such as laparoscopic surgery large tissue masses such as the uterus are removed through small incisions, decreasing post-operative pain and hospitalization time.

Several types of hysterectomy are performed fully or partially laparoscopically, and these include Total Laparoscopic Hysterectomy (TLH) where the uterus and cervix are removed through few small incisions made in the abdomen; Laparoscopic Supracervical Hysterectomy (LSH) where the uterus is removed, but the cervix is left intact. In both cases the uterus is removed through one of the small incisions using an instrument called a morcellator. Another approach is a Total Vaginal Hysterectomy (TVH) where the uterus and/or cervix are removed through the vagina.

In laparoscopic hysterectomies for example, the uterus is removed using instruments inserted through small tubes into the abdomen, resulting in few small incisions in the abdomen. A laparoscopic approach offers surgeons better visualization of affected structures (e.g., by using an endoscope) than either vaginal or abdominal hysterectomy.

There remains a need to resecting and/or removing tissue from the interior of an organ while maintaining a surface of the organ to prevent tissue from being removed from spreading within the body. Such procedures and devices require an ability for the medical practitioner to be aware of the position of the cutting device relative to the surface of the tissue of the organ while the device is within the organ. This would allow the physician to remove a significant portion of the tissue within the organ and remove the organ from the body. Such devices and systems can be used in any part of the body, with a hysterectomy being one example.

### SUMMARY

The present disclosure includes systems and methods for resecting and/or removing tissue from the interior of an organ and monitoring a proximity of the tissue removal device to a surface of the organ to prevent the surface of the organ from being cut or breached by the cutting device. In some variations, the cutting device advances through the outer surface of the organ when inserted into a cavity within the organ. In alternate variations, the device is introduced through an opening of the organ. The devices and methods described herein are explained with respect to performing a hysterectomy. However, the methods, devices, and systems can be used in any body location unless otherwise specifically claimed.

In one example, the prevent disclosure teaches a system for resecting tissue within an interior of an organ. Such a variation can include a probe having a proximal portion and a distal portion; a cutting member configured to remove tissue and located at the distal portion of the probe; at least one sensor located adjacent to the cutting member, the sensor configured to generate a signal comprising an environmental condition adjacent to the cutting member; and a controller configured to receive the signal of the environmental signal and use the signal to determine whether the cutting member is adjacent to an exterior surface of the organ.

The sensor can comprise a mechanism selected from a group consisting of a capacitance sensing mechanism, an impedance sensing mechanism, an optical sensing mechanism, and an ultrasound mechanism.

In one variation of the system, the controller is configured to generate an alert signal upon detecting that the cutting member is adjacent to the exterior surface of the organ. Such an alert signal can comprise an aural alert, a visible alert, a tactile alert, and a combination thereof.

The probe and cutting mechanism can comprise a mechanical or an electrosurgical-based cutting mechanism. In certain variations, the sensor is located adjacent to the cutting mechanism or adjacent to a window or opening in the probe that exposes the cutting member.

In variations where the cutting mechanism comprises an electrosurgical cutting mechanism, the cutter can comprise an electrode element, a resistively heated element, an inductively heated element, an ultrasound transmission element, and a light energy transmission element.

The controller of the present system can include an algorithm for de-activating the cutting member in response to the signal that the cutting member is within a pre-determined proximity to the organ surface. The algorithm can also modulate the speed that the cutting member removes tissue.

The systems described herein can further comprise a negative pressure source in fluid communication with the probe and cutting mechanism, where the negative pressure source extracts resected tissue through a passageway in the probe. Alternatively or in combination, the systems can comprise a positive pressure source in fluid communication with the probe.

The present disclosure also includes methods for resecting tissue. In one such variation, the method can comprise introducing a probe into an interior of an organ, wherein a working end of the probe includes a cutter and sensor mechanism adjacent to the cutter, where the sensor mechanism is configured to detect a surface of the organ; resecting tissue with the cutter generating a signal with the sensor mechanism when the sensor mechanism detects the cutter approaching the organ surface from the interior of the organ; and removing a substantial volume of the organ from within the interior of the organ without the cutter perforating the organ surface from the interior thereby preventing dispersion of potentially malignant tissue.

The method can further include variations where the sensor mechanism comprises at least one of a capacitance sensing mechanism, an impedance sensing mechanism, an optical sensing mechanism and an ultrasound mechanism. In an additional variation, the sensor mechanism is operatively coupled to a controller to provide signals consisting of at least one of aural, visible, or tactile signals.

The method can also include a controller that employs an algorithm for de-activating the cutter in response to a signal that the cutter is within a pre-determined proximity to the organ surface. The de-activating step can comprise stopping movement of the cutter or stopping energy delivery to the cutter. In additional variations, the controller includes an algorithm for modulating the speed of movement of the cutter in response to the signal that the cutter is within a pre-determined proximity to the organ surface.

Variations of the method can also include mobilizing the organ with the intact organ surface after the substantial volume is removed and removing the organ from the patient's body.

In an additional variation, a method of resecting tissue comprises introducing a probe into an interior of an organ, wherein a probe working end includes a first sensor component; disposing a second sensor component at an exterior surface of the organ; and activating the probe to resect tissue wherein the first and second sensor components cooperate to provide at least one signal indicating a proximity of the probe to the exterior surface of the organ. At least one of the sensor components comprises a component selected from the group consisting of a capacitance sensing mechanism, an impedance sensing mechanism, an optical sensing mechanism and an ultrasound mechanism and the other sensor component cooperates to enhance a sensitivity of said signals/

The sensor component can include a gas, liquid or gel disposed at the exterior of the organ. Alternatively, or in combination the second sensor component comprises a sac disposed at the exterior of the organ.

In an additional variation, the present disclosure includes methods for laparoscopic hysterectomy. For example, the method can include introducing a probe into a uterine cavity, wherein a probe working end includes a sensor mechanism for sensing the proximity of the cutter to an exterior surface of a uterine wall; activating the probe to resect tissue from within the uterine cavity wherein the sensor mechanism provides signals indicating the proximity of the cutter to said exterior surface; and removing a substantial volume of the tissue from within the uterine cavity without perforating the uterine wall thereby preventing dispersion of potentially malignant uterine tissue. The method can include sealing and/or ligating blood vessels communicating with the uterus.

The method can further comprise removing a substantial volume of the tissue within the uterine cavity without perforating the uterine wall from within the cavity such that the uterine wall forms an intact shell. The method can also include transecting the shell of the uterine wall away from the patient's body.

The methods and/or devices described herein can be performed in a supracervical procedure, a trans-vaginal approach, an endoscopic approach, or in an open surgical approach.

In an additional variation, a method of resecting at least a portion of an organ can include isolating the tissue mass or organ from its blood supply; introducing a resecting probe into the organ, wherein a probe working end includes a cutter and sensor mechanism for sensing the proximity of the cutter to a surface of the organ; activating the cutter to resect tissue wherein the sensor mechanism provides signals indicating the proximity of the cutter to the organ surface; and removing a substantial volume of the organ without perforating the organ surface thereby preventing dispersion of potentially malignant tissue.

### BRIEF DESCRIPTION OF THE DRAWINGS

FIG. 1 is a schematic view of a tissue resection device and block diagram of operating components corresponding to the invention for use in a laparoscopic resection procedure.

FIG. 2 is a perspective view of the working end of a resection device of the type shown in FIG. 1 showing a sensor mechanism carried by the working end.

FIG. 3A is a schematic view of the patient's uterus and abdominal region showing initial steps of a laparoscopic hysterectomy procedure.

FIG. 3B is a sagittal view of the patient's uterus and abdominal cavity showing another step in a method of the invention comprising introducing a trocar through the uterine wall from the abdominal cavity.

FIG. 3C is a sagittal view of a subsequent step in a method of the invention comprising introducing the resecting device into the interior of the uterus, actuating the device to reset tissue and removing tissue through passageway in the resecting device.

FIG. 3D is a sagittal view of a further step in a method of the invention comprising introducing utilizing the resecting device to reset and remove a substantial volume of the interior of the uterus while sensor mechanisms indicate and/or control when a cutting member comes into proximity to the wall of the uterus.

FIG. 4 is a sagittal view of the patient's uterus and abdominal cavity showing the variation in the method wherein a sensor responsive media is applied around the exterior surface of the uterus.

FIG. 5 is another sagittal view of the patient's uterus and abdominal cavity showing another variation in the method wherein a sensor responsive mesh sac is disposed around the exterior surface of the uterus.

FIG. 6 is another variation of the invention showing a resection device including an ultrasound sensor array carried at the working end of the device.

FIG. 7 is an enlarged perspective view of the working end of the device of FIG. 6.

FIG. 8A is a schematic view of an alternative working end similar to that of FIG. 7 with piezoelectric transducers in a different configuration.

FIG. 8B is a schematic view of another working end similar to that of FIGS. 7 and 8A with piezoelectric transducers in a different configuration.

FIG. 9A is a schematic view of a patient's uterus showing an initial step in a hysterectomy procedure using the resection device of FIGS. 6 and 7.

FIG. 9B illustrates a subsequent step in the method of FIG. 9A where the working end resects tissue in the fundus of the uterus with the ultrasound sensor system controlling the limits of resection.

FIG. 9C shows another subsequent step that includes articulating the working end of the resection device.

FIG. 9D is yet another step in the method showing the working end re-oriented to resect tissue in a different location.

FIG. 10 is a view of a robotic system of the invention where a robotic arm operates the resection device of FIGS. 6 and 7.

FIG. 11 is a schematic view of a resection procedure similar to that of FIGS. 9A-9D utilizing an additional ultrasound sensing system at an exterior of the patient's body to provide additional images of the outer surface of the uterus.

FIG. 12 is a schematic view of another robotic system similar to that of FIG. 10 where the first robotic arm operates the resection device, and a second robotic arm operates the ultrasound sensor at the exterior of the patient's body of the type shown in FIG. 11.

FIG. 13 is another device corresponding to the invention that comprises a treatment tool with a movable sleeve that fits over the elongated shaft of the treatment tool, where the moveable sleeve carries an image sensor for capturing visual images of the treatment site.

FIG. 14 is a view of the working end of a treatment device and moveable sleeve of FIG. 13.

### DETAILED DESCRIPTION OF THE INVENTION

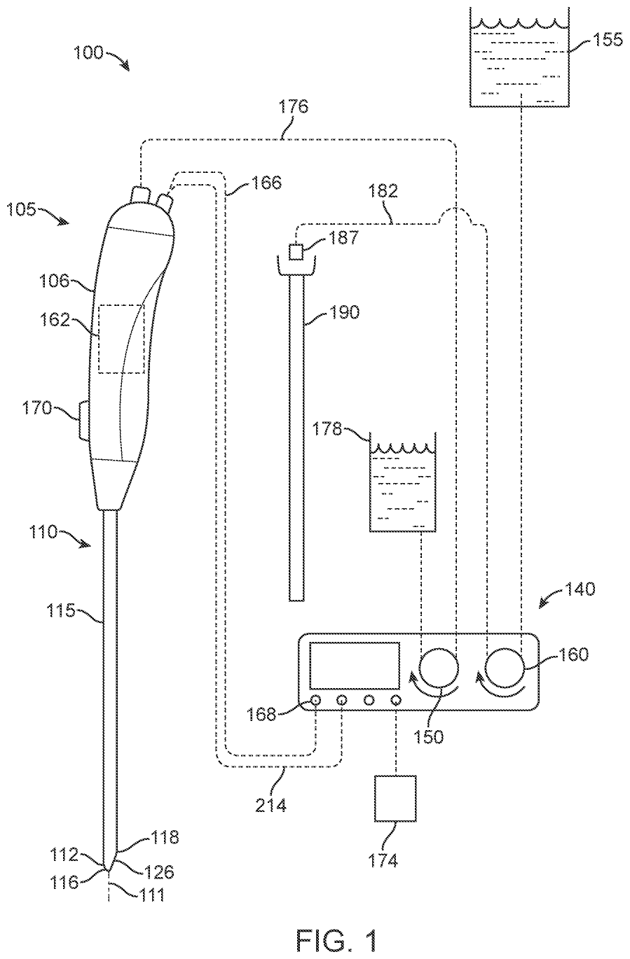

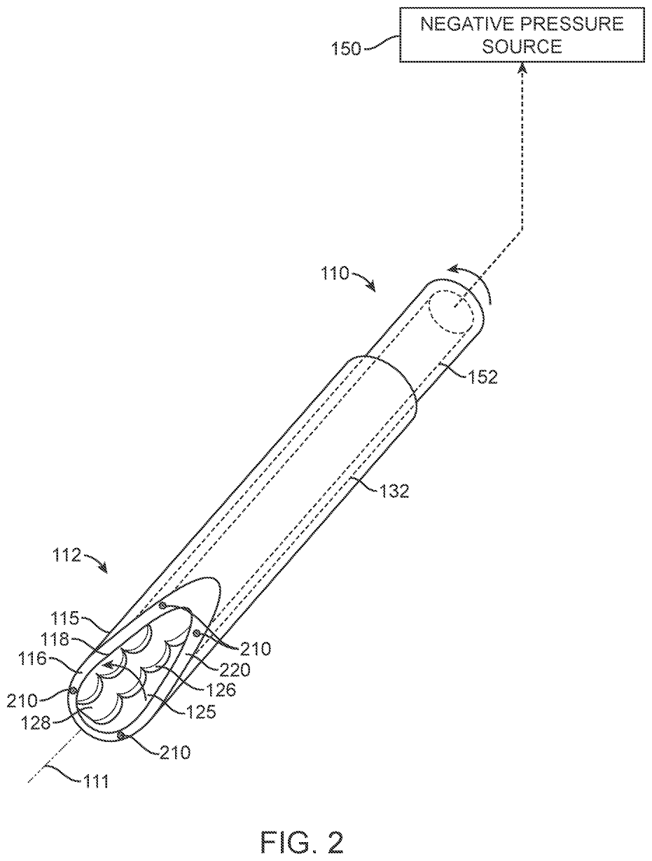

FIGS. 1 and 2 illustrate a tissue resection system 100 that includes a handheld single-use tissue cutting device or resection device 105. The device 105 has a handle portion 106 that is coupled to a shaft portion 110 having an outer diameter ranging from about 3 mm to 20 mm. The shaft portion 110 extends along axis 111 and can have a length suitable for introducing directly into a body space or into an organ, for example, introducing through a trocar in a laparoscopic procedure or for introducing through a working channel of an endoscope.

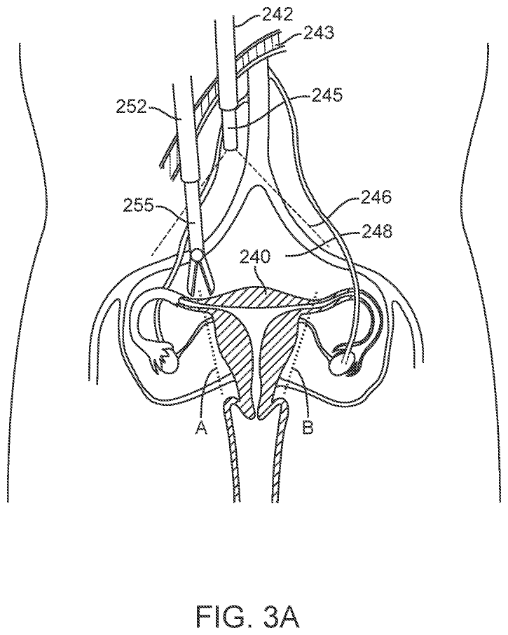

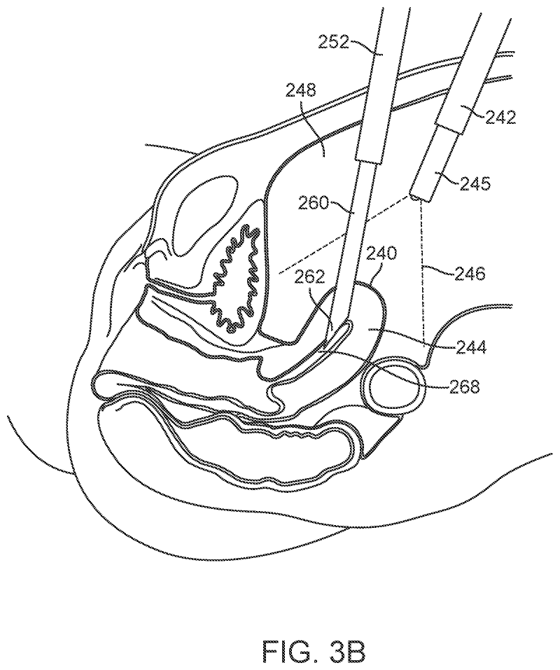

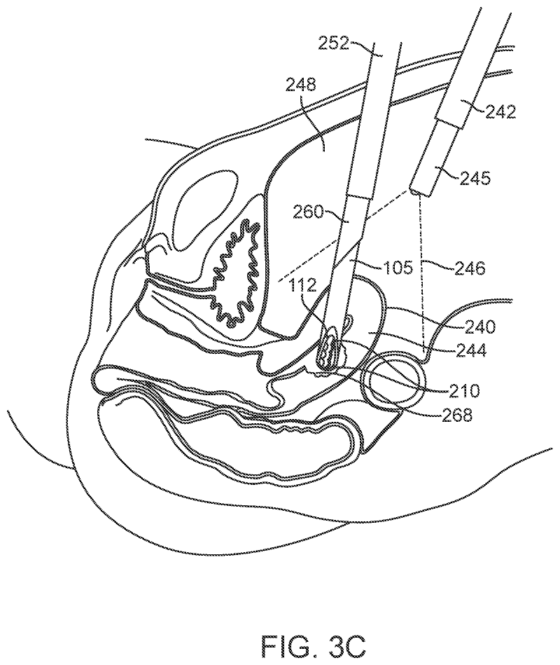

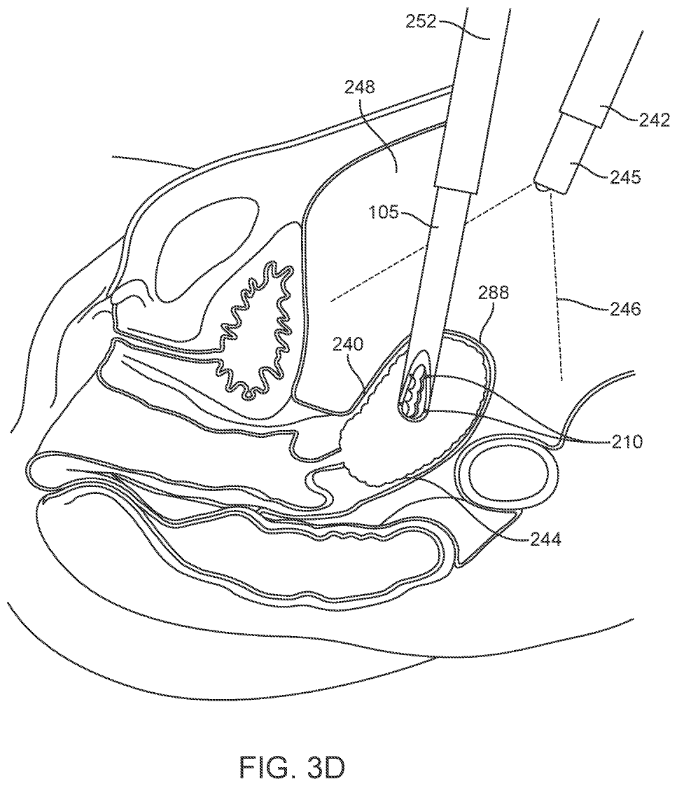

In one variation, a handheld resecting device 105 as depicted in FIGS. 1 and 2 can be used to perform a laparoscopic hysterectomy procedure as depicted in FIGS. 3A to 3D. Referring to FIGS. 1 and 2, the resection device 105 is a tubular cutter as is known in the art with a shaft portion 110 and working end 112. The shaft 110 comprises an assembly of a first or outer sleeve 115 extending along axis 111 to a distal end 116 having a window 118 therein for receiving tissue. A second or inner sleeve 125 with a distal blade edge 126 and distal opening 128 is dimensioned to rotate in bore 132 of outer sleeve 115. The outer and inner sleeves, 115 and 125, are typically fabricated of thin-wall stainless steel but any other suitable materials can be used. As can be understood from FIGS. 1-2, rotation of the inner sleeve 125 will cut tissue captured in the window 118 of the outer sleeve. FIG. 2 shows the working end 112 of the assembly of outer sleeve 115 and inner sleeve 125 with the inner sleeve 125 rotating and in a partially window-open position.

As can be seen in FIG. 1-2, the resection system 100 includes a controller 140 that is adapted for (i) controlling a motor drive in the resecting device 105 as will be described below; (ii) controlling at least one sensor system carried by the resection device 105 that will be described further below, (iii) controlling a negative pressure source or outflow pump 150 operatively coupled to a tissue extraction channel 152 in the resection device 105, and (iv) controlling an optional fluid source 155 and inflow pump 160 for distending or flooding a treatment site with a fluid, such as saline.

Referring to FIG. 1, the controller 140 includes algorithms for driving a motor 162 in the handle 106 of the resecting device 105. The motor can be a brushless DC motor and controller 140 can be configured to operate the motor at a preset RPM or a user-selected RPM between 100 and 2,000 RPM. FIG. 1 shows an electrical cable 166 extending from connector 168 in the controller 140 to the resecting device handle 106. The resecting device 105 can be operated by a switch 170 in the handle 106 or a footswitch indicated that 174 coupled to the controller 140.

Still referring to FIG. 1, the controller 140 includes a roller pump 150 that provides a negative pressure source for extracting tissue through the passageway 152 in the resecting device 105. The roller pump 150 in combination with the flexible tubing 176, is configured to pump fluid and extracted tissue chips through the tubing into the collection reservoir 178.

Again, referring to FIG. 1, controller 140 has a second roller pump 160 adapted to provide fluid flows into a site targeted for resection. A fluid source 155 is coupled to a flexible fluid infusion tubing 182 that is engaged by the roller pump 160 and that further extends to a fitting on cannula 190, which is adapted for access to the treatment site. The cannula 190 can be inserted into the site and can be used as an access pathway for the resection device 105, or the cannula can be used for fluid infusion independent of the resection device. In another variation, the fluid infusion tubing 182 can be coupled to the resection device 105 so that fluid flows to the working end 112 and window 118 in a path in the annular space between the outer sleeve 115 and the inner sleeve 125.

Now turning to FIG. 2, the working end 112 of the resecting device 105 is shown in an enlarged perspective view. In one variation shown in FIG. 2, a sensor system is shown disposed around the cutting window 118 in the working end. This variation shows four capacitance sensors 210 disposed around the window 118, which comprise the distal termination of paired wire leads as is known in the art capacitance sensors. The capacitance sensors 210 are coupled to the controller 140 through cable 214 (FIG. 1). The sensors 210 can be carried in a thin polymeric coating 220 on the outer sleeve 125. In this embodiment, there are four capacitance sensors, but there could be from 1 to 20 sensors on the outer sleeve 125. In another variation, one or more capacitance sensors could be carried on the inner sleeve surface opposing the sharp blade edges. As will be described below, capacitive sensors 210 can provide a signal to the user when the cutting blade 126 (FIG. 2) approaches the periphery of an organ targeted for resection. While FIG. 2 shows a variation of the resecting device 105 with capacitance sensors 210, it should be appreciated that other types of sensors can be used to determine the proximity of the cutting blade to an organ periphery, such as optical sensors, impedance sensors, magnetic sensors, and the like.

Now turning to FIGS. 3A to 3D, a method corresponding to the invention is described relating to the resection of a uterus in a new form of laparoscopic hysterectomy. FIG. 3A is a schematic view of the patient's abdominal cavity and a uterus 240 targeted for resection. In a first step of the method, a first sleeve or cannula 242 is introduced through the abdominal wall 244, and an endoscope 245 is inserted through the sleeve to provide a field of view 246 in the abdominal cavity 248.

FIG. 3A further shows a second cannula 252 introduced through the abdominal wall 244, after which a cutting-sealing device 255, such as electrosurgical cutting and sealing device, is introduced through the cannula 252 for use in sealing and transecting blood vessels communicating with the uterus 240. As is known in the art of performing a laparoscopic hysterectomy, the uterine arteries are sealed and transected, and the broad ligaments, fallopian tubes, and fascia are transected along lines A and B to mobilize the uterus 240. Thereafter, the cutting-sealing device 255 is withdrawn from cannula 252.

FIG. 3B depicts a subsequent step of the method wherein a sharp trocar sleeve 260 is introduced through the second cannula 252 by the physician, and then, under laparoscopic vision, the distal tip 262 of the trocar sleeve 260 is advanced through the uterine wall 244 into the uterine cavity 268.

FIG. 3C shows the next step in the method wherein the resection device 105 is introduced through the cannula 252 and trocar sleeve 260 into the interior of the uterus 240, and thereafter the trocar sleeve 260 is withdrawn, leaving the working end 112 of the resection device 105 within the interior of uterus 240. In one variation of the method, the fluid source 155 and infusion tubing 182 are coupled to the resection device 105 to provide a fluid flow into the uterine cavity 268 through the annular space between the outer sleeve 115 and the inner sleeve 125 (see FIGS. 1-2). By this means, the uterine cavity 268 can be distended to some extent, while the controlled fluid inflow assists in the resecting procedure and further assists in the extraction of tissue chips from the site. In another variation (not shown), a cervical seal member can be introduced trans-vaginally to seal the uterine cavity 268, wherein the cervical seal can be a probe shaft, an inflatable member, or other types of seals known in the art. In another variation, the fluid source 155 and infusion tubing 182 can be coupled to a trans-cervical probe and seal (not shown) to provide a fluid flow into the uterine cavity 268.

Still referring to FIG. 3C, the physician then can actuate the resecting device 105 to resect tissue in a blind method while observing the exterior of the uterus 240 with the endoscope 245. The physician can manipulate the working end 112 of the resecting device 105 to core out the interior of the uterus 240 while leaving the uterine wall 244 completely intact. It can now be seen that the purpose of the capacitance sensors 210 is to provide signals to indicate the proximity of the cutting blade 126 to the exterior of the uterine wall 244. As indicated in FIG. 3D, in one variation, the capacitance sensors 210 can sense a change in tissue capacitance when the window 118 and blade move close to the exterior of the uterine wall 244. The plurality of capacitive sensors 210, as shown in FIG. 2 allows for sensing proximity to the surface of the uterine wall no matter the orientation of the working end 112. The resecting procedure can be considered complete when the physician has removed a substantial volume of tissue from the interior of the uterus 240 and, in effect, leaves only a shell 288 if the uterus is left in place, as shown in FIG. 3D. By this means, it can be understood that no resected tissue, and thus no potentially malignant tissue, has been exposed outside of the interior of the uterus 240. Rather, all tissue has been resected and immediately extracted through passageway 152 in the inner sleeve 125 and then collected in the collection chamber 178 with no possibility of contaminating the abdominal cavity 248. In one aspect of the method, at least 50%, at least 60%, at least 70%, at least 80%, or at least 90% of the mass of the uterus 240 is resected and extracted to leave a reduced-volume shell 288 of the uterus (FIG. 3D). Following the resection and extraction of the bulk of the uterus 240, the reduced-volume shell 288 of the uterus can be removed in methods known as in a conventional supracervical or other laparoscopic hysterectomy procedure. Typically, the reduced-volume uterine shell 288 can be removed intact in a transvaginal approach.

During the resection steps described above, the controller 140 can modulate fluid inflows to and from the site by controlling the roller pumps. The flow rates into and out of the uterine cavity 268 can be from 10 mL/min to 1000 mL/min and can be modulated depending on a cutting speed selected by the physician.

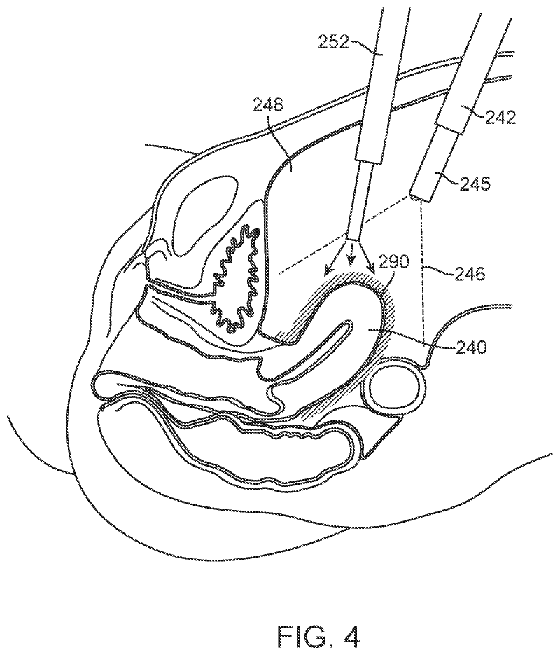

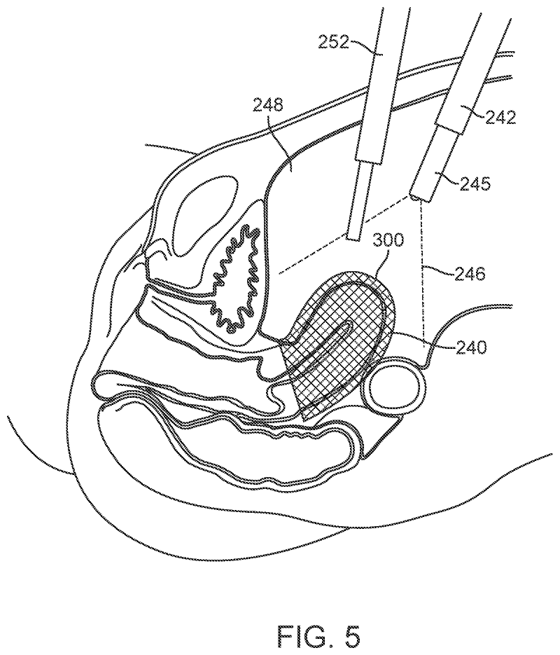

In another embodiment in another variation shown in FIG. 4, a sensor enhancing media may be sprayed, painted, flooded, or otherwise disposed around the exterior of the uterus 240 to enhance the sensitivity of the capacitance sensors 210 or other sensing mechanisms. For example, FIG. 4 illustrates a conductive gel 290 that may be sprayed or painted onto the exterior of the mobilized uterus 240 which will increase the resolution of the capacitive sensors 210. Such a media 290 can be a conductive gel, such as a hypertonic saline gel. A similar conductive gel would enhance the resolution impedance sensors. In another variation, a magnetic sensitive material could be disposed around the uterus 240, which could increase the resolution of a magnetic sensor carried by the working end 112 of the resecting device 105. In another variation shown in FIG. 5, a mesh net 300 can be disposed around the uterus 240 for similar purposes. For example, a structure similar to that stretchable nylon stocking with conductive threads could be disposed around the uterus 240 to increase the sensitivity of a capacitance sensor 210, an impedance sensor, or a magnetic sensor.

In another variation, light emitters may be disposed in one or more locations around the window 118 of the exterior sleeve 125. Such light emitters can be added to the device of FIG. 2 or can be used instead of capacitance sensors 210 or other sensors. The light emitters can be a distal end of one or more optical fibers, for example. It can be understood that the physician can then see the brightness of the light through the translucent uterine wall and understand the proximity of the cutting blade 126 to the wall surface.

In one variation, the controller 140 includes algorithms to modulate or terminate operation of the resecting device 105 when the capacitance sensors 210 or other sensor mechanisms indicate the proximity of the cutting blade to the exterior of uterine wall 244. In another variation, the sensor system can provide warning signals to the position of the cutting blade in the form of aural, visual, or tactile signals.

By using the system and method described above, it can be understood that the laparoscopic hysterectomy can be performed without the risk of dispersing any potentially malignant tissue in the abdominal cavity 248. All resected tissue chips are maintained within the interior of the uterus 240, with the uterine wall itself functioning as a containment sac. The system and method can be performed with any type of resecting device, such as a mechanical cutter as shown herein, in which a blade can cut by rotation, reciprocation, or both. In other variations, the resecting device may be an RF device, ultrasound device, laser device, microwave device, resistive heat device, or the like.

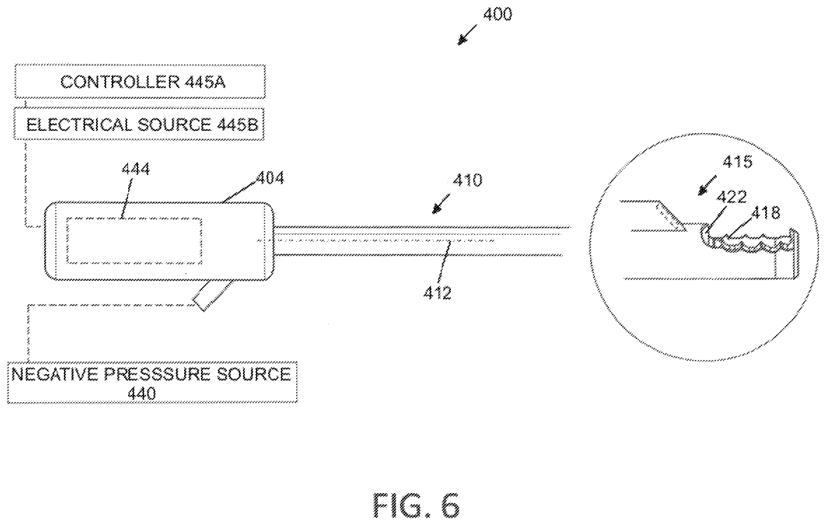

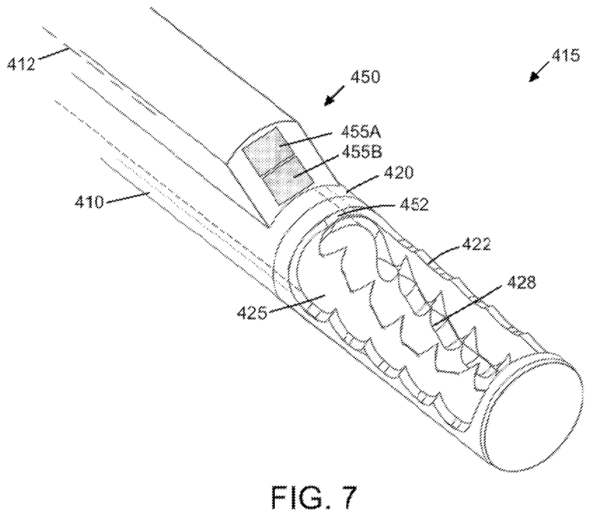

Now turning to FIG. 6, another variation of the invention is shown, which again includes a handheld surgical resection device 400 with a handle 404 coupled to an elongated shaft 410 that extends about axis 412 to a distal end working end 415 that is configured with a tissue cutting mechanism or resecting component 418. FIG. 7 is an enlarged perspective view of working end 415 of FIG. 6, which comprises an outer sleeve 420 with a tissue receiving window 422 and a rotating inner sleeve 425 with cutting window 428 therein that is rotated at high speed to resect tissue. A negative pressure source 440 is coupled to an interior lumen 442 in the inner sleeve 425 to suction tissue into the outer sleeve window 422 and inner sleeve window 428 to capture tissue for resection. A motor 444 in the handle 404 is operated by a controller 445A and electrical source 445B to rotate or oscillate the inner sleeve 425 at a high speed, for example, from 500 RPM to 15,000 RPM, as is known in the art. It should be appreciated that the tissue resecting component can comprise a mechanical resecting device, an electrosurgical resecting device, or an electrosurgical ablation device.

FIG. 7 illustrates an ultrasound sensor 450 is carried on a surface of the shaft 410 located proximally from the proximal end 452 of window 422 in the outer sleeve 420. In one variation, the sensor 450 can comprise a plurality of ultrasound transducers and is shown with two piezoelectric transducers 455a and 455b that are oriented to propagate sound waves axially and angularly relative to axis 412 and outer and inner sleeve windows 422 and 428. The ultrasound sensor typically uses a frequency ranging from 2 MHz to 10 MHz.

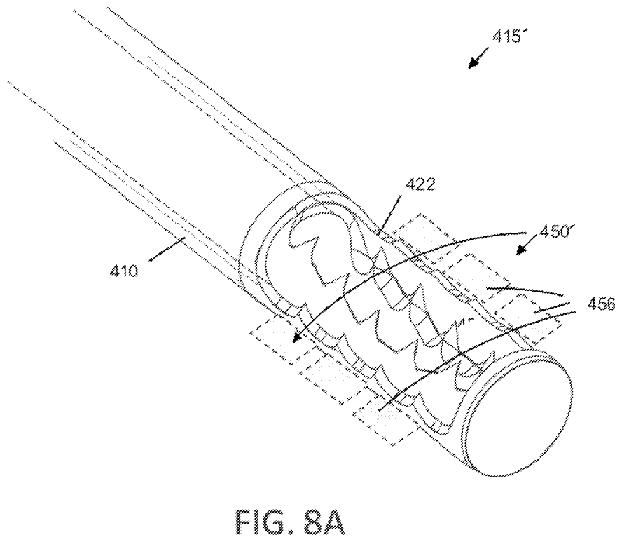

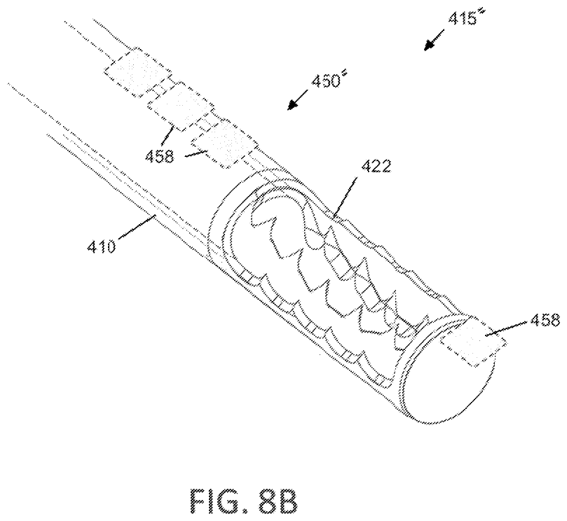

Now turning to FIGS. 8A and 8B, other similar variations of working ends 415′ and 415″ are shown, which function in a similar manner the working end 415 of FIGS. 6 and 7. FIG. 8A schematically illustrates the working end 415′ with an ultrasound sensor assembly 450′ comprising a series of piezoelectric transducers 456 positioned on each side of the outer sleeve window 422. Any form of suitable housing (not shown) can be provided to house such transducers 456. In FIG. 8B, the working end 415″ illustrates an ultrasound sensor 450″ comprising piezoelectric transducers 458 positioned both proximal and distal from the outer sleeve window 422.

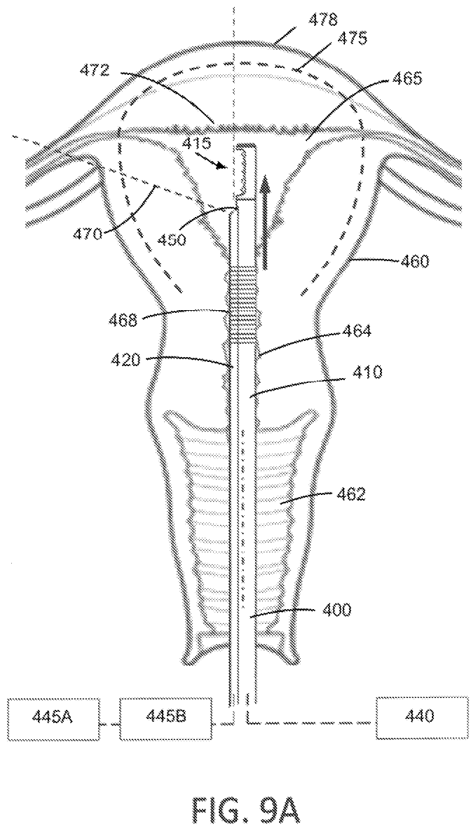

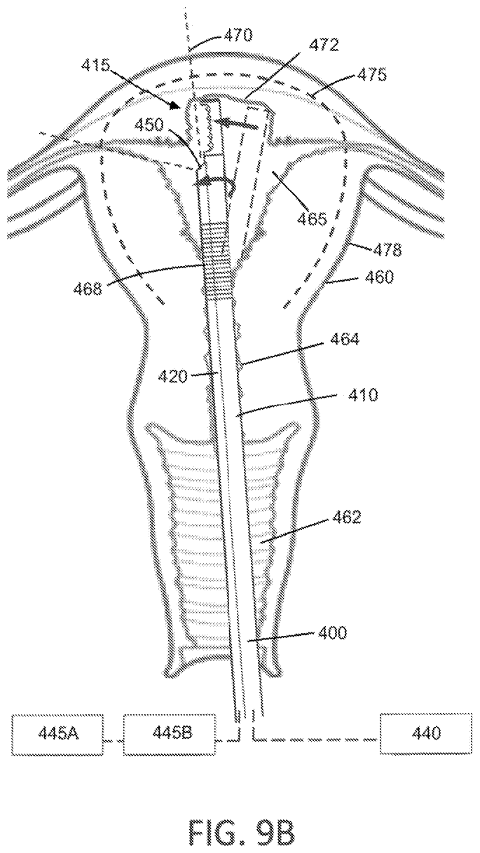

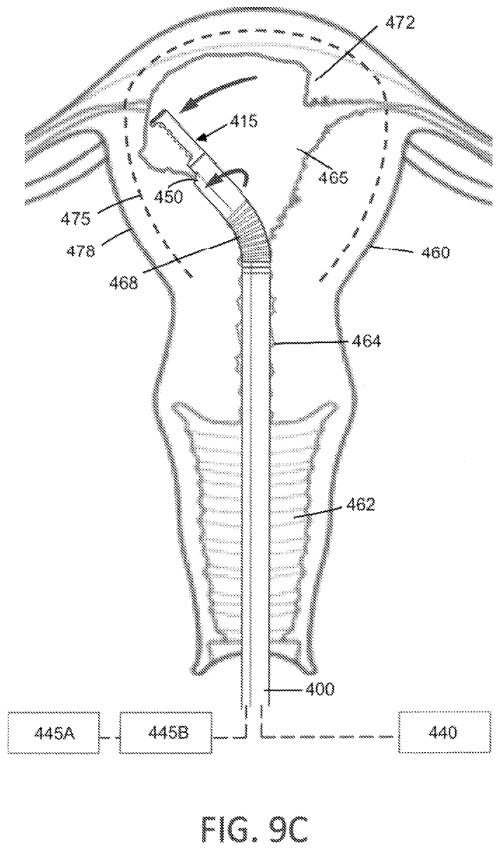

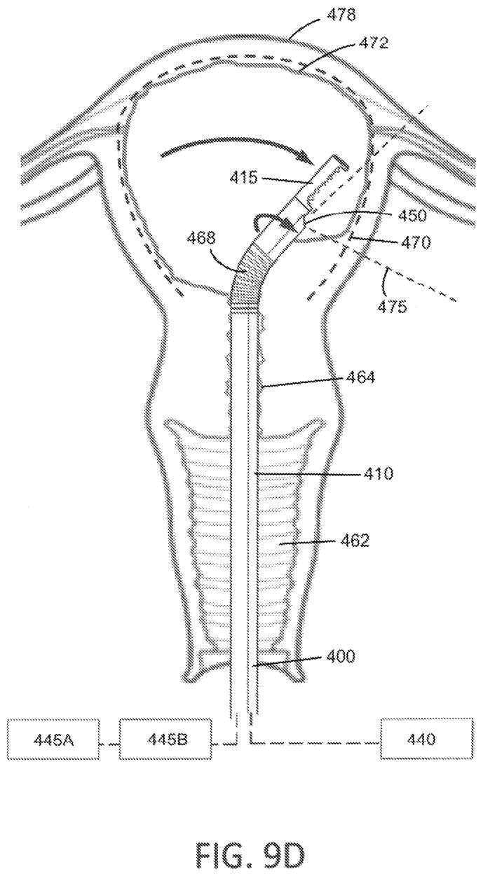

FIGS. 9A-9D schematically illustrate a patient's uterus 460 and a method of using the resection device of FIGS. 6 and 7 in a form of supracervical hysterectomy procedure. In FIG. 9A, the shaft 410 of the resection device 400 is introduced through the patient's vagina 462 and endocervical canal 464 into a uterine cavity 465. The shaft 410 includes an outer sleeve 420 with an articulating section 468 in which the inner sleeve is rotated by a flexible drive shaft as is known in the art. FIG. 9A shows the angle of propagation 470 of ultrasound waves from the sensor 450 carried by the working end 415. Now turning to FIG. 9B, it can be seen how the physician's movement of the shaft 410 axially, angularly, and rotationally 415 can be used to move cutting widows 422, 428 across the fundus 472 of the uterine cavity 465 to resect and remove tissue. In FIGS. 9A and 9B, the dashed line indicates a calculated “resection limit” 475 where the ultrasound sensor 450 and controller 440A determine that the exterior surface 478 of the uterus 460 is a selected distance from the working end 415 and wherein the controller delivers a warning or stops actuation of the resection device 400.

FIG. 9C illustrates another step of the method where the physician articulates the articulating section 468 and then again moves the shaft 410 and working end 415 axially, angularly, and rotationally 415 to orient the cutting widows 422, 428 in a selected direction to resect tissue from a first lateral side 484a of the uterine cavity 465. At all times during such a resecting step, the ultrasound sensor 450 and controller 440A are adapted to warn the physician or terminate actuation of the resection device when tissue cutting approaches the resection limit 475. FIG. 9D illustrates the articulated working end 415 being adjusted to resect tissue on the opposing or second lateral side 484b of the uterine cavity 465. Following the resection step illustrated in FIGS. 9A-9D, the bulk of the uterus will be resected, morcellated, and removed, leaving only the serosa 488 of the uterus intact. Thereafter, remaining serosa 488 can be resected and removed trans-vaginally or laparoscopically, and the tissue region around the endocervical canal 464 is sealed as is known in the art of performing a supracervical hysterectomy.

In another variation, the medical system includes a fluid source controlled by the controller for providing a controlled fluid inflow to the treatment site. In a variation, the controller is configured to maintain a selected set pressure in the treatment site. In another variation, the controller is configured to adjust the set pressure in response to an operating condition of the procedure, which can be at least one of (i) the volume of resected tissue, (ii) the number of times the controller de-activated the tissue resecting component, (iii) the change in sensed pressure in the site, (iv) the rate of change of sensed pressure in the site, and (v) total time of activation of the tissue resecting component.

In general, the medical system for resecting tissue in a body of a patient comprises a probe having an elongated shaft assembly extending about a longitudinal axis to a working end, where the working end is adapted for introduction into a site in an interior of an organ, a tissue resecting component within the working end, an ultrasound sensor carried by the working end, and a controller coupled to the tissue resecting component and the ultrasound sensor for sending imaging signals to the controller which locate the surface of the organ, and where the controller in response to the imaging signals, is configured to (i) determine a distance between the tissue resecting component and the surface of the organ; (ii) allow activation of the tissue resecting component when positioned at least a selected distance from said surface of the organ; and (iii) modulate activation of the tissue resecting component when positioned at less than said selected distance from said surface of the organ.

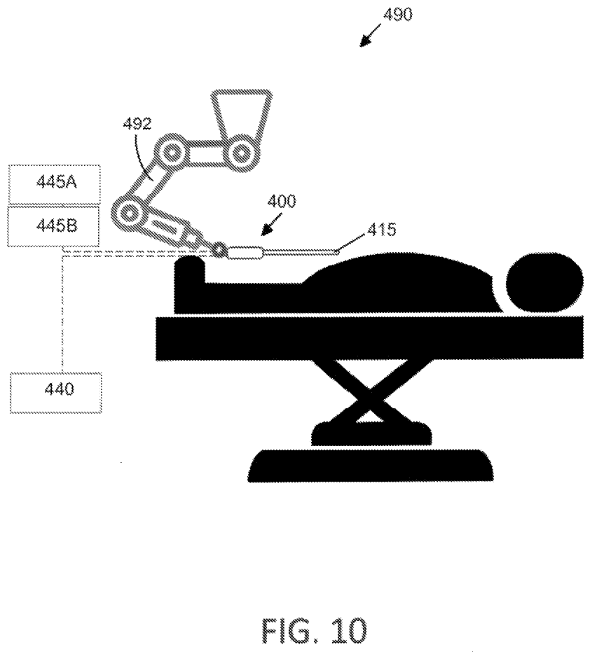

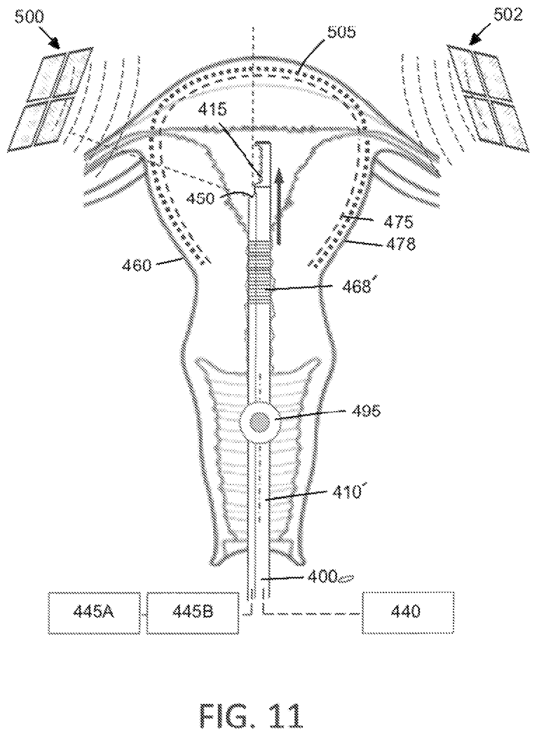

FIG. 10 is a schematic view of another variation of the invention which depicts a robotic system 490 that includes a multi-joint robotic arm 492 that can move in multiple directions, where such a robotic system can carry the resection device 400 of FIGS. 6 and 7, or at least a working end 415 as shown in FIGS. 7 and 11, to perform a surgical procedure, for example, a procedure of the type shown in FIGS. 9A-9D. In FIG. 11, the shaft 410′ is shown with a robotically articulated joint 495 as well as a robotically-controlled articulating section 68′ closer to the working end 415. In FIGS. 10 and 11, it can be understood that a physician can move the multiple articulating elements of the robotic arm 492 to move the working end 415 axially, angularly, rotationally, as well as articulating the working end 415, as shown in FIGS. 9C-9D to robotically remove tissue in a hysterectomy procedure.

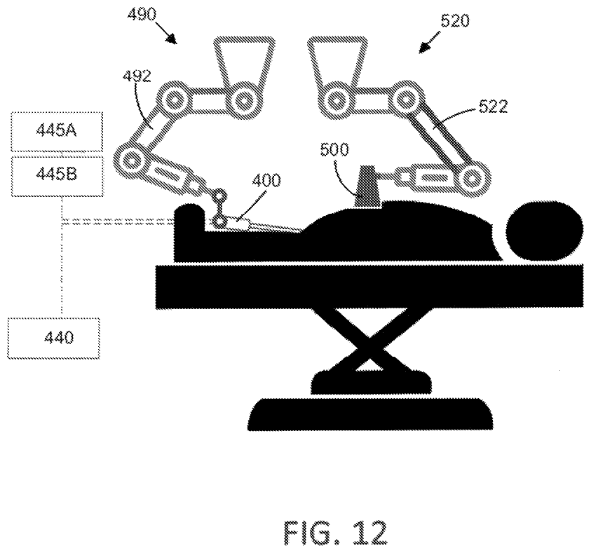

In FIG. 11, another optional component of the system 490 of FIG. 10 is shown, which comprises at least one additional ultrasound sensor 500 disposed outside the patient's body to image the targeted treatment site, which in this instance is the patient's uterus 460. In the variation of FIG. 10, two ultrasound sensors, 500 and 502, are shown and are adapted to provide another layer of safety by detecting and displaying the outer surface of the uterus 460 and providing a second calculated “resection limit” 505 where the ultrasound sensors 500, 502 and controller 445A determine the location of the exterior surface 478 of the uterus 460 and define the resection limit as a selected distance inward from surface 478. In this variation, the signals from ultrasound sensor 450 of the device 400 of FIGS. 6-7 and the first resection limit 475 can be integrated with, or compared to, the calculations provided by the external ultrasound sensors 500, 502 for monitoring and managing the operation of the resection device relative to the exterior surface 478 of the uterus 460. FIG. 12 shows another variation of the invention where a second robotic system 520 with robotic arm 522 that is configured to carry the ultrasound sensor 500 of FIG. 11. The controller 445A can be programmed to move the robotic arm 522 and sensor 500 in coordination with the controlled movement of the first robotic arm 492 and resection device 400. In addition, the second robotic arm 522 can carry a reservoir of ultrasound gel and a fluid delivery mechanism to apply the ultrasound gel to the patient's skin under and around the ultrasound sensor 500.

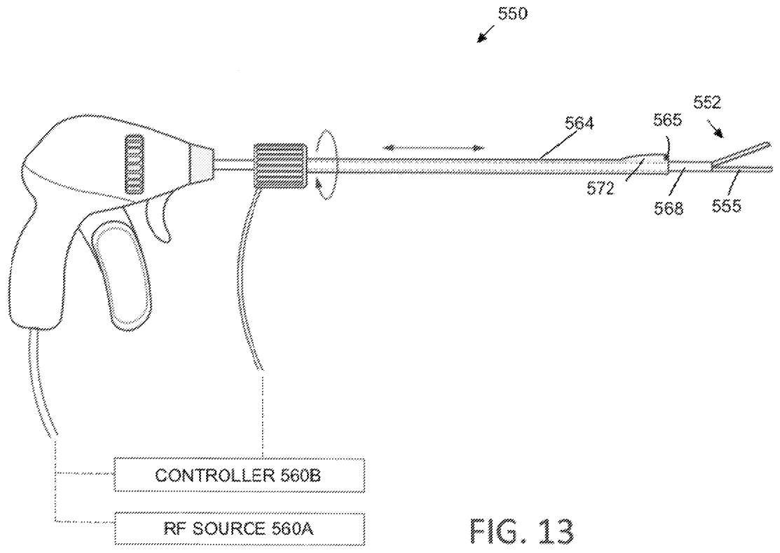

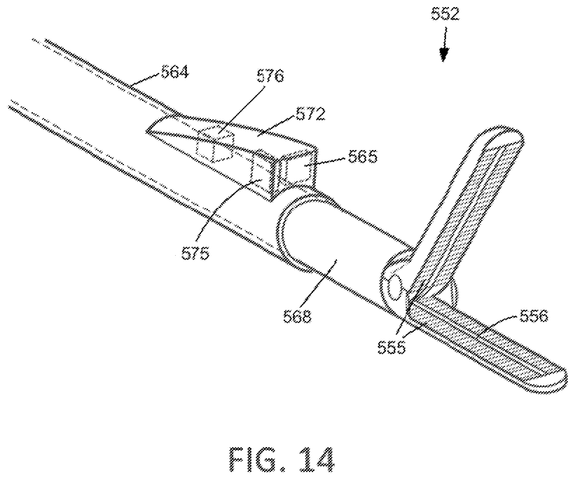

FIGS. 13 and 14 show another variation of a handheld treatment device 550 that in one variation has a working end 552 with jaws comprising a tissue sealing and cutting mechanism as is known in the art, for example, with an RF electrode arrangement 555 and a blade (not visible) that can be advanced in slot 556 in the jaws when closed (see FIG. 14). The device 550 is coupled to RF source 560A and controller 560B. In this variation, an additional outer sleeve 564 is provided and carries a distal image sensor 565. The outer sleeve 564 is adapted for positioning over the elongated the shaft 568 of the treatment tool 550. In this variation, the image sensor 565 is coupled to controller 560B, which includes an imaging processor that is adapted for displaying video images on a display (not shown). The outer sleeve 564 is moveable axially and rotationally to allow the physician to observe the treatment site as the working end 552 of the treatment tool 550 is moved.

FIG. 14 is an enlarged view of the working end 552 of the device 550 of FIG. 13, where it can be seen that the distal section 572 of the movable outer sleeve 564 carries the image sensor 565, as well as an, LED 575 for illuminating the targeted site. In addition, the distal section 572 of the sleeve 564 carries an accelerometer 576 for sending the controller 560B signals of the rotational and angular position of the working end 552, which then is used to create a stable, “upright” image on the video display. In other words, the accelerometer 576 can rotate and maintain the images in an upright orientation on a display no matter the rotational movement of the outer sleeve 564 and image sensor 565.

It should be appreciated that the treatment device 550 of FIG. 13 can consist of a grasper, a tissue sealer, a tissue cutter, a biopsy device, a morcellation device, an ablation device, and a stapler. In another variation, the treatment device 550 can further include a robotic mechanism as described above coupled to the outer sleeve 564 configured to move the sleeve rotationally relative to the shaft. In another variation, a robotic mechanism is coupled to the tissue treatment device 550 and the moveable outer sleeve 564, where the robotic mechanism is configured to move the treatment device in at least one direction selected from rotationally, axially, angularly, or a combination thereof, and where the robotic mechanism is further configured to move the sleeve 564 rotationally relative to the shaft.

What is claimed is:

1. A medical system for resecting tissue in a body of a patient, comprising:
a probe having an elongated shaft assembly extending about a longitudinal axis to a working end, where the working end is adapted for introduction into a site in an interior of an organ;
a tissue resecting component within the working end;
an ultrasound sensor carried by the working end; and
a controller coupled to the tissue resecting component and the ultrasound sensor;
where the ultrasound sensor sends imaging signals to the controller which locate a surface of the organ; and
where the controller in response to the imaging signals, is configured to:
(i) determine a distance between the tissue resecting component and the surface of the organ;
(ii) allow activation of the tissue resecting component when positioned at least a selected distance from said surface of the organ; and
(iii) modulate activation of the tissue resecting component when positioned at less than said selected distance from said surface of the organ.

2. The medical system of claim 1 where the tissue resecting component comprises at least one of a mechanical resecting device, an electrosurgical resecting device and an electrosurgical ablation device.

3. The medical system of claim 2 where the controller is configured to de-activate the tissue resecting component.

4. The medical system of claim 2 where the controller is configured to slow a movement of a mechanical resecting device.

5. The medical system of claim 1 where the ultrasound sensor uses a frequency ranging from 2 MHz to 10 MHz.

6. The medical system of claim 1 where an imaging angle of the ultrasound sensor is partly transverse to the longitudinal axis of the elongated shaft assembly.

7. The medical system of claim 1 where the tissue resecting component comprises a first sleeve and a second sleeve concentrically located where the first sleeve has a tissue-receiving window and the second sleeve is moveable to resect tissue received in the tissue-receiving window.

8. The medical system of claim 7 where the second sleeve is motor driven.

9. The medical system of claim 7 where the second sleeve is configured to move at least one of by rotation, by axial reciprocation and by oscillation.

10. The medical system of claim 1 further comprising a negative pressure source controlled by the controller adapted to aspirate resected tissue through the tissue resecting component and the elongated shaft assembly.

11. The medical system of claim 1 further comprising a fluid source controlled by the controller for controlling a fluid inflow to the site.

12. The medical system of claim 11 where the controller is configured to control at least one of negative pressure source and the fluid source to control fluid pressure in the site.

13. The medical system of claim 11 where the controller is configured to maintain a selected set pressure in the site.

14. The medical system of claim 11 where the controller is configured to adjust a set selected pressure in response to an operating condition.

15. The medical system of claim 14 where the operating condition is at least one of (i) a volume of resected tissue, (ii) a number of times the controller de-activated the tissue resecting component, (iii) a change in sensed pressure in the site, (iv) a rate of change of sensed pressure in the site, and (v) total time of activation of the tissue resecting component.

16. A medical device for resecting tissue, comprising:
an elongated shaft extending about an axis to a working end configured with a resecting tool for resecting tissue;
an ultrasound sensor carried by the elongated shaft;
a controller operatively coupled to the resecting tool and the ultrasound sensor; and
a display adapted to display ultrasound images generated from the ultrasound sensor; and
a robotic mechanism coupled to the elongated shaft configured to move the resecting tool in at least one direction selected from rotationally, axially, angularly or a combination thereof.

17. The medical device of claim 16 wherein the elongated shaft includes at least one articulating portion.

18. The medical device of claim 17 wherein the robotic mechanism is configured to articulate the at least one articulating portion.

19. The medical device of claim 16 wherein resecting tool comprises an outer sleeve coaxial with an inner sleeve and having respective outer and inner windows and wherein the inner sleeve is rotatable in the outer sleeve to resect tissue.

20. The medical device of claim 16 wherein the controller is configured to receive signals from the ultrasound sensor and determine when the resecting tool is near an exterior surface of an organ.

21. The medical device of claim 16 where the ultrasound sensor comprises a plurality of piezoelectric transducers.

22. The medical device of claim 16 further comprising a fluid source controlled by the controller for controlling a fluid inflow to a site.

23. The medical device of claim 22 where the controller is configured to control at least one of negative pressure source and the fluid source to control fluid pressure at a treatment site.

24. The medical device of claim 22 where the controller is configured to maintain a selected set pressure in the site.

25. The medical device of claim 22 where the controller is configured to adjust the selected set pressure in response to an operating condition of a procedure.

26. The medical device of claim 25 where the operating condition is at least one of (i) a volume of resected tissue, (ii) a number of times the controller de-activated the resecting tool, (iii) a change in sensed pressure in the site, (iv) a rate of change of sensed pressure in the site, and (v) a total time of activation of the resecting tool.

27. A medical device, comprising:
an elongated sleeve extending about an axis to a working end, where the elongated sleeve is configured for rotational movement over a shaft of a tissue treatment device; and
an image sensor carried at the working end of the elongated sleeve;
where the tissue treatment device consists of at least one of a grasper, a tissue sealer, a tissue cutter, a biopsy device, a morcellation device, an ablation device and a stapler.

28. The medical device of claim 27 where the elongated sleeve is configured for axial movement over the shaft of the tissue treatment device.

29. The medical device of claim 27 where the elongated sleeve is detachable from the tissue treatment device.

30. The medical device of claim 27 where the elongated sleeve is integral to the tissue treatment device.

31. The medical device of claim 27 further comprising at least one LED carried at the working end of the elongated sleeve.

32. The medical device of claim 27 further comprising at least one accelerometer carried by the elongated sleeve.

33. The medical device of claim 27 further comprising a robotic mechanism coupled to the elongated sleeve configured to move the elongated sleeve rotationally relative to the shaft.

34. The medical device of claim 33 further comprising a robotic mechanism coupled to the tissue treatment device and the elongated sleeve, where the robotic mechanism is configured to move the tissue treatment device in at least one direction selected from rotationally, axially, angularly or a combination thereof, and where the robotic mechanism is further configured to move the elongated sleeve rotationally relative to the shaft.
